(19) United States
(12) Patent Application Publication
(10) Pub. No.: US 2023/0390535 A1
(43) Pub. Date: Dec. 7, 2023

(54) GUIDE EXTENSION CATHETERS

(71) Applicant: Cardiovascular Systems, Inc., St. Paul, MN (US)

(72) Inventors: Bruce H. Asmus, Minnetonka, MN (US); Jeffrey A. McBroom, Champlin, MN (US); Jared W. Connelly, Minneapolis, MN (US); Craig Stowell, Watertown, MN (US); Christopher J. Gotz, Maple Grove, MN (US); Daniel J. Vreeman, Otsego, MN (US); Kristen M. Rauscher, Lino Lakes, MN (US)

(21) Appl. No.: 17/809,668

(22) Filed: Jun. 29, 2022

Related U.S. Application Data
(60) Provisional application No. 63/365,715, filed on Jun. 2, 2022.

(51) Int. Cl. A61M 25/09; A61M 25/00; A61M 25/01

(52) U.S. Cl. CPC A61M 25/0905; A61M 25/0052; A61M 25/0138; A61M 25/0102; A61M 25/0023; A61M 2025/0024; A61M 2025/09141

(57) ABSTRACT

A guide extension catheter comprising a proximal reinforcing section that is completely surrounded by, or encased within, a polymer covering or coating the proximal reinforcing section, and further comprising an exemplary semi-circular flared section disposed proximal to the proximal reinforcing section, wherein the flare is interrupted by a flattened bottom. The proximal reinforcing section is configured to interconnect with a flat ribbon which is connected with or defines a proximally extending push rod. The proximal reinforcing section does not extend beyond the proximal-most end of the exemplary semi-circular flared section

### CROSS-REFERENCE TO RELATED APPLICATIONS

This application claims priority to U.S. provisional application No. 63/365,715, filed Jun. 2, 2022 and titled HIGH PERFORMANCE MICROCATHETERS, the entire contents of which is incorporated herein by reference.

### STATEMENT REGARDING FEDERALLY SPONSORED RESEARCH OR DEVELOPMENT

Not applicable

### BACKGROUND OF THE INVENTION

### Field of the Invention

Guide extension catheters for use in intravascular medical procedures.

### Description of the Related Art

Generally, arteries may become occluded or otherwise compromised by, e.g., loss of elasticity and/or compliance as a result of a build-up of calcified material along the artery's lumen and/or reaching within the artery wall. Percutaneous intervention and treatment may be achieved using known interventional tools using known techniques such as, without limitation, angioplasty, atherectomy, lithotripsy and/or thrombectomy as well as procedures relating to crossing chronic total occlusions and delivering stents. Guide catheters may be used to assist in treating coronary arteries or peripheral arteries. In some cases, percutaneous access may be achieved at a patient's femoral artery and in other cases at a patient's radial artery (transradial). Facilitating interventional and/or diagnostic tool delivery is a common use of the guide catheter. Once positioned within the vasculature, a guide catheter may be used to facilitate a smoother advancement of an interventional tool and/or a percutaneous catheter through tortuous vasculature by mitigating radial deflection, buckling and/or kinking of the interventional tool and/or percutaneous catheter. In addition to the complex percutaneous interventional issues described above that require additional support for a guide catheter, transradial procedures may also require additional support. Guide extensions may be used to provide the required support in each case.

Commonly used guide extension systems are available in 5F, 5.5F, 6F, 7F and 8F, including Guideliner® catheter from Teleflex, Guidezilla® and Guidezilla II® from Boston Scientific Corp, Telescope® from Medtronic, Boosting Catheter from QXMedical, and Guidion® from Interventional Medical Device Solutions.

Generally, these known systems comprise a proximal portion, or rapid exchange section, that is partially open, i.e, not fully enclosed. For example, Guideliner® and Guidion® each comprise a half-pipe portion at a proximal end of an otherwise fully enclosed distal portion and Guidezilla® comprises a tapered or skived open portion at the proximal end of an otherwise fully enclosed distal portion. Telescope® comprises an open tapered or skived proximal portion leading to an open half-pipe proximally extending portion. QXMedical's Boosting Catheter also comprises a completely circumferentially flared skived or tapered proximal portion that leads to an open half-pipe portion.

To further illustrate some of these known designs, FIG. 1 shows the commercially available guide extension catheter device 10 marketed as Guidezilla®, illustrated with polymer coating removed, and which comprises a braided flexible tube 12, a skived or tapered proximal section 14 that is directly connected to a push rod or rail 16 at a lower portion 15 of the skived or tapered proximal section 14 and further comprising a proximal handle (not shown) connected with the push rod or rail 16 to enable an operator to translate and/or rotate the guide extension catheter.

FIG. 2 illustrates the commercially available guide extension catheter device 20 marketed as Guideliner® comprising an overcoated braided flexible tube 22, a side view of a skived or tapered portion 24 and a half-pipe portion 26, and a push rod 28 connected with a lower portion 27 of half-pipe portion 26 and further comprising a proximal handle (not shown) connected with push rod 28 to enable an operator to translate and/or rotate the guide extension catheter.

Note that the prior art device of FIG. 1 does not comprise the half-pipe section of the device shown in FIG. 2. Further, the known device of FIG. 1 provides the skived or tapered proximal section that is open, i.e., not surrounded by a polymer coating or jacket. Similarly, the known device of FIG. 2 provides the skived or tapered portion and half-pipe portions as open, i.e., not surrounded by a polymer coating or jacket. However, both devices shown in FIGS. 1 and 2 comprise a polymer overcoating or jacket around the braided flexible tube portions.

It is desired to provide a guide extension catheter with improved control of various characteristics of the tube, e.g., steerability, variable bending flexibility along the working length, pushability, and/or collapse or kink resistance.

Further, the open skived/tapered and/or half-pipe designs of the known commercialized systems may not form a seal between the fully enclosed portion of the guide extension catheter and the surrounding guide catheter. Moreover, known guide extension systems comprise a proximal portion, or rapid exchange section, opening that interfaces or connects with a curvilinear half-pipe section for loading and/or exchanging catheters or interventional tools and the like. This can result in difficulty in inserting the catheter and/or tool into the lumen of the distal fully enclosed portion. Additionally, the braided flexible tube provides the backbone to the desired characteristics of the guide extension system. If the tube is too flexible the positioning of the guide extension system within the anatomy could be difficult due to the low pushability and cause the guide extension system to be placed more proximal than desired.

The various inventions disclosed herein address these, inter alia, issues.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a known guide extension catheter.

FIG. 2 illustrates another known guide extension catheter

FIG. 3 illustrates a side view of one embodiment of the present invention.

FIG. 4A illustrates a side partial cutaway view of one embodiment of the present invention.

FIG. 4B illustrates a top partial cutaway view of one embodiment of the present invention.

FIG. 5 illustrates a side partial cutaway view of one embodiment of the present invention.

FIG. 6 illustrates a side partial cutaway view of one embodiment of the present invention.

FIG. 7 illustrates a side partial cutaway view of one embodiment of the present invention.

FIG. 8 illustrates a side partial cutaway view of one embodiment of the present invention.

FIG. 8A illustrates a top perspective view of one embodiment of the present invention.

FIG. 9A illustrates a perspective cutaway view of one embodiment of the present invention.

FIG. 9B illustrates a perspective cutaway view of one embodiment of the present invention.

FIG. 9C illustrates a perspective cutaway view of one embodiment of the present invention.

FIG. 9D illustrates a perspective cutaway view of one embodiment of the present invention.

FIG. 10A illustrates a perspective cutaway view of one embodiment of the present invention.

FIG. 10B illustrates a perspective cutaway view of one embodiment of the present invention.

FIG. 10C illustrates a perspective cutaway view of one embodiment of the present invention.

FIG. 10D illustrates a perspective cutaway view of one embodiment of the present invention.

FIG. 11A illustrates a perspective cutaway view of one embodiment of the present invention.

FIG. 11B illustrates a perspective cutaway view of one embodiment of the present invention.

FIG. 11C illustrates a perspective cutaway view of one embodiment of the present invention.

FIG. 11D illustrates a perspective cutaway view of one embodiment of the present invention.

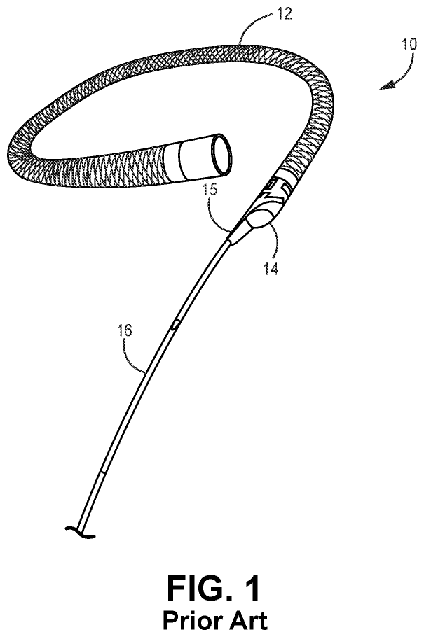

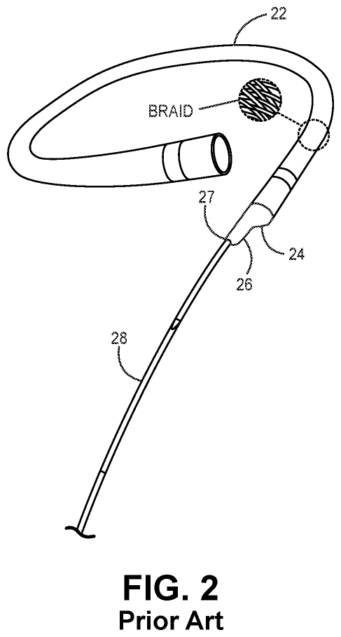

### DETAILED DESCRIPTION OF THE INVENTION

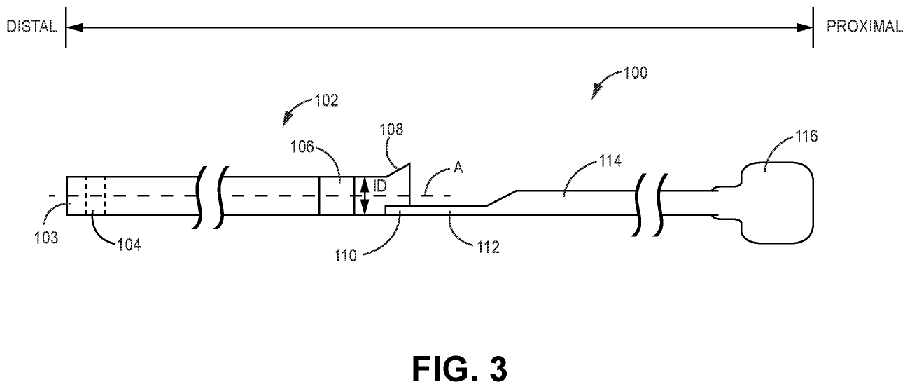

With reference to the Figures, FIG. 3 illustrates one embodiment of a guide extension catheter 100 of the present invention. Guide extension catheter 100 thus comprises a flexible tube section 102 that is shown covered with a material such as, e.g., and without limitation, a polymer, e.g., PTFE or the like. The flexibility of the flexible tube section 102 may vary or change, e.g., increase in the distal direction or proximal direction, or may be constant along the length of the flexible tube section. A flexible tip 103 is disposed at the distal end of the flexible tube section 102 and a radiopaque marker 104 may be disposed at or near a distal end and/or a proximal end of the flexible tube section 102. As shown, distal radiopaque marker 104 is just proximal the tip 103. Another radiopaque marker 106 may be provided at or near a proximal end of the flexible tube section 102. Both of the distal and proximal radiopaque markers 104, 106 are shown as “bands”, though other marker configurations such as radiopaque deposits or coating are also contemplated and within the scope of the present invention.

At least a part of flexible tube section 102 may be formed, or reinforced by, a structure that provides the tubular shape and the desired flexibility and other operational characteristics. For example, a laser-cut hypotube, a braid and/or oppositely wound coils may be employed, with an outer poly covering, coating or jacket as described above.

In some embodiments of the present invention, the flexible, reinforced tube section 102 and/or a proximal portion that may not be reinforced, may transition to a proximally positioned semi-circular section that may comprise a tapering, partial cone or flare, or may comprise a partial cylinder, wherein the semi-circular shape of the proximally positioned semi-circular section is interrupted by a flattened bottom section which will be discussed further infra.

In other embodiments, the flexible, reinforced tube section 102 may transition to a proximally positioned complete tapering, partial cone or flare, or a complete cylinder wherein the semi-circular shape of the proximally positioned semi-circular section is not interrupted by a flattened bottom section which will be discussed further infra. In some embodiments, the flattened bottom section may comprise a component that is manufactured as flat and does not require the component to start is a non-flat shape and then be flattened.

In the illustrated embodiment of FIG. 3, the flexible, reinforced tube section 102 transitions at a proximal end region into a semi-circular section comprising a partial, or interrupted, flared, or partial cone-shaped, section 108, wherein the inner and outer diameters of the partially flared section 108 are tapering and becoming larger moving in the proximal direction, and wherein the inner and outer diameters of the flexible, reinforced tube section 102 are smaller than the inner and outer diameters at any point along the partial flared section 108. As shown in FIG. 3, the partially flared section 108 is an interrupted or incomplete or asymmetric flare or cone that comprises a flattened lower surface 110 so that the partially flared section 108 this comprises a semi-circular structure with a linear or flat surface lower surface 110 at any longitudinal cross section of partially flared section 108 and is not, therefore, a complete flare or cone shaped structure. The partially flared section 108 terminates at a proximal end at a substantially perpendicular angle to a central longitudinal axis A, without tapering or skive.

The flattened lower surface 110 is shaped to be complementary to a flat ribbon section 112 that is at least partially disposed along the flattened lower surface 108 of the partial flared section 108, and therefore within the confines of, or surrounded by, the partial flared section 108 and, in some embodiments, may also extend a distance distally into and along the flexible tube section 102 which may extend proximally up to the partially flared section 108.

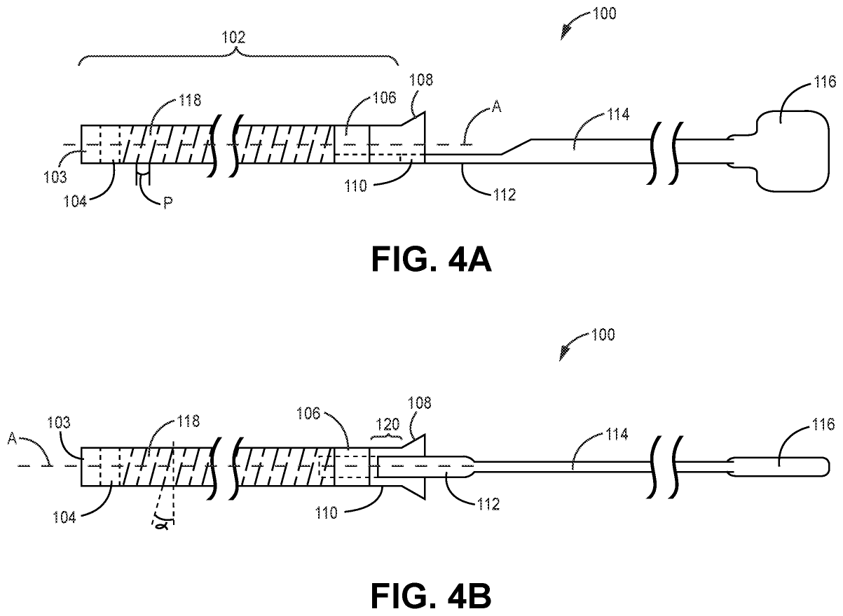

Flat ribbon section 112 may be operationally connected, e.g., welded or soldered or snap-fit, with a proximal, or proximally extending portion, of the tube section 102. As shown, the flat ribbon section 112 may be configured to extend along the flattened lower surface 110 and slightly into the inner diameter ID of the partial flared section 108. In other embodiments the flat ribbon section 112 may occupy a space within the polymer in order to not impact the inner diameter ID of at least the partial flared section 108. Flat ribbon section 112 may be operationally connected, e.g., welded or soldered or snap-fit, with a proximal, or proximally extending portion, of the flexible tube section 102. Similarly, in embodiments comprising the flat ribbon section 112 extending distally past the partial flared section 108 into the flexible tube section 102, the flat ribbon section 102 may reduce the nominal ID of the flexible tube. Dashed lines in FIG. 4A extending the length of the flat ribbon section 112 distally to operatively connect with the exemplary laser-cut tube illustrates one such embodiment.

In other embodiments, the flat ribbon section 112 may be disposed within a wall W, e.g., encased by polymer, of the partial flared section 108 and, in some embodiments as discussed above into a wall W′ of the flexible tube section 102, in order to not reduce the nominal inner diameter ID of the partial flared section 108 or, again in some embodiments, the nominal inner diameter of the flexible tube section 102. As seen in FIGS. 4A and 4B, the flat ribbon section 112 may be positioned at a distal end of a push rod 114 which may comprise a handle 116 disposed at a proximal end of the push rod 114. An operator may manipulate the handle 116 which is external to the patient during an intravascular procedure, in order to translate and/or rotate the guide extension device 100 and, in particular, the position of the device 100 within a patient's vasculature.

FIGS. 4A and 4B illustrate one embodiment of the flexible, reinforced tube section 102 formed by a spiral laser-cut tube 118. As shown, and with continued reference to FIG. 3, the pitch (or distance between cuts in the longitudinal direction, or along central axis A corresponding with a central longitudinal axis of the lumen (not shown but is defined through the tube section 102 and partial flared section 108) P are constant as is the angle of the spiral cuts a, relative to a line perpendicular to axis A as shown in FIG. 4B, along the length of the flexible, reinforced tube section 102.

The spiral laser-cut tube 110 forming the flexible, reinforced tube section 102 of FIGS. 4A and 4B is illustrated as comprising a distal marker band 104 and a proximal marker band 106 with an additional proximally-extending portion 120 of the flexible tube and semi-circular section, shown as partial flare 108 is illustrated as not comprising the laser-cut tube or hypotube structure. In other words, one embodiment may comprise a proximal portion that may comprise a proximal portion 120 of the flexible tube 102 and the more proximal partial flared section 108 both being formed from a polymer, e.g., PTFE, or related material that is operatively connected with the laser-cut tube 118 and/or an intervening radiopaque marker 106 as shown, wherein the reinforcing structure, e.g., laser-cut tube 110 does not extend proximally into the proximal portion 120 and/or the proximal partial flared section 108. In other embodiments, the spiral laser-cut tube 118 may extend through the sections comprising proximal and/or distal radiopaque markers 104, 106, and/or the more proximal flexible tube section 120 and/or through at least a portion of the partial flared section 108 to provide shaping and/or flexible or other support of the proximal section 120 of the flexible tube section 102 and/or partial flared section 108.

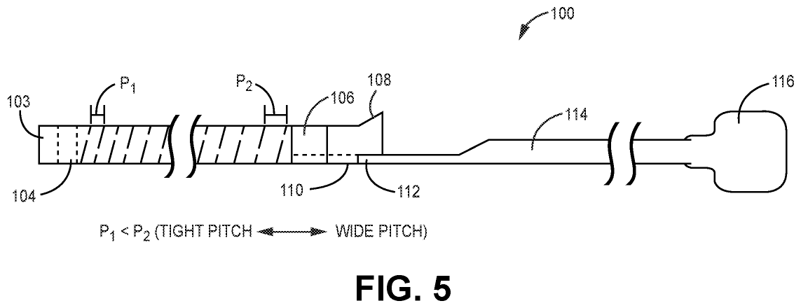

FIG. 5 illustrates an embodiment of an exemplary guide extension catheter 100 comprising a flexible tube section 102 formed by the spiral laser-cut tube 118′ of FIGS. 4A and 4B, wherein the pitch, or longitudinal distance between cuts, P, shown as P1 which is smaller in length than P2, increases moving from the distal end to the proximal end of the spiral laser-cut tube 118′ in order to modify the flexibility profile along the length of the spiral laser-cut tube 118′ and flexible tube section 102. Another embodiment may comprise increasing or decreasing the angle α of the cuts along the spiral laser-cut tube, shown in FIG. 4B, relative to the central axis A.

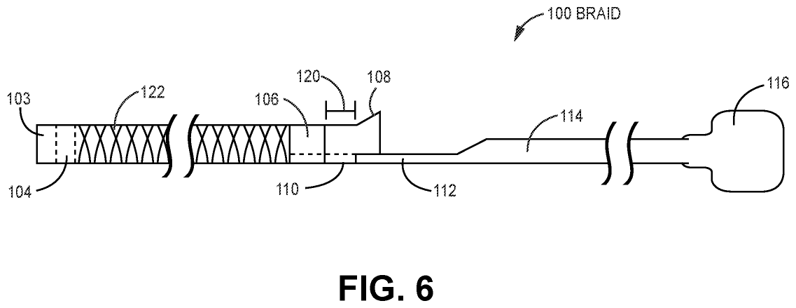

FIG. 6 illustrates a guide extension catheter 100 BRAID embodiment similar to that of FIGS. 4A and 4B, but instead of a spiral laser-cut tube forming the flexible tube section 102, a braided structure 122 is provided. As is known in the art, the braid pitch and/or pic numbers may be selected as constant along the length of the flexible tube, or may be modified to adjust or affect the longitudinal flexibility, and other characteristics described above, of the flexible tube.

Moreover, at least one portion of the flexible tube section 102 may comprise a spiral laser-cut tube 118 and/or 118′ as described above in connection with FIGS. 4A, 4B and 5, while at least one other portion of the flexible tube section 102 may comprise a braided structure 122 in order to adjust and fine tune the operational characteristics of the flexible tube section 102.

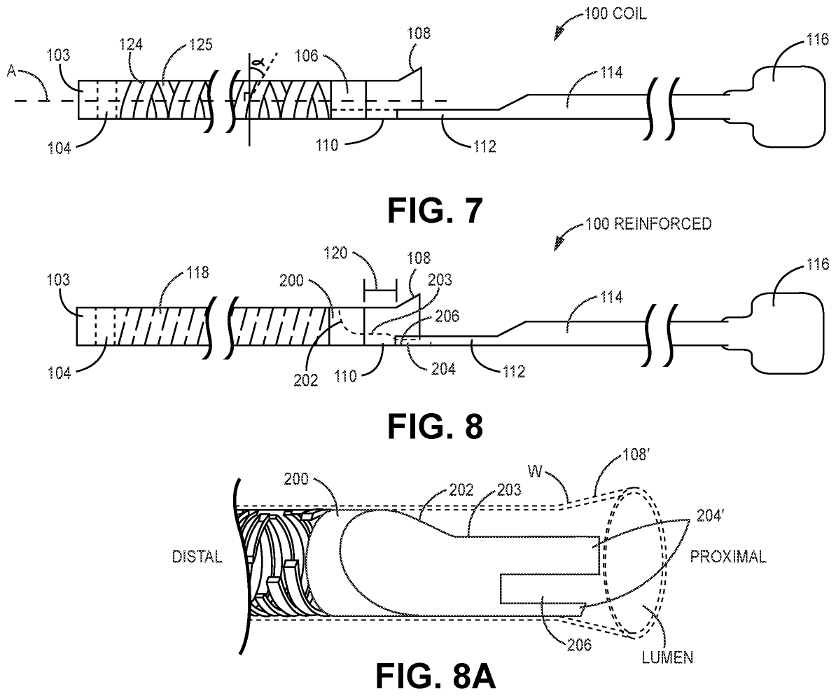

FIG. 7 also illustrates a guide extension catheter 100 COIL embodiment similar to that of FIGS. 4A and 4B, but instead of a spiral laser-cut tube 118, 118′ and/or a braid 122 as described above, the flexible tube section 102 may be comprised of two oppositely-wound coils 124, 125, wherein an upper coil 125 may be wound over a lower coil 124. The artisan will recognize that two or more, e.g., 3, oppositely wound coils may be employed in this manner. For example, a first lower coil may be wound over a liner tube 126 and a second intermediate coil wound in an opposing direction over the first lower coil and a third upper coil may be wound in an opposing direction relative to the second intermediate coil's winding direction. The coil wind pitch (longitudinal distance between wires within a coil) and/or the angle of the coil wind relative to a line a that is perpendicular to the central axis A, may be selected as constant along the length of the flexible tube section 102, or may be modified to adjust or affect the longitudinal flexibility, and other characteristics described above, of the flexible tube section 102. The wind pitch for any given coil at any point along the length of a wound coil, e.g., 124, 125 may be 0 or greater than 0 and may vary along the longitudinal length of the wound coil. Accordingly, the flexibility may be modified, e.g., increase in the distal direction, along the length of flexible tube section 102.

Moreover, at least one portion of the flexible tube section 102 may comprise a spiral laser-cut tube 118 and/or 118′ as described above in connection with FIGS. 4A, 4B and 5, and at least one other portion of the flexible tube 102 may comprise two or more oppositely wound coils, e.g., 124, 125, and/or at least one other portion of the flexible tube may comprise a braid 120 in order to adjust the operational characteristics of the flexible tube section 102.

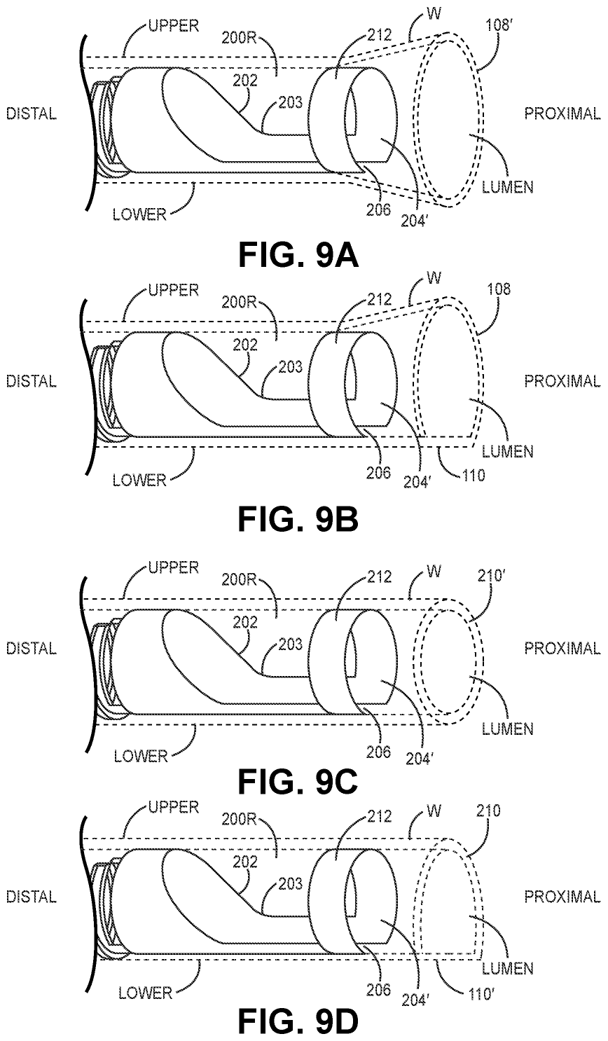

Turning now to FIG. 8, an alternative, and preferred, guide extension catheter 100 REINFORCED embodiment is provided, similar to that described above in connection with FIGS. 4A-7, but with a proximal reinforcing region 200 (shown with dashed lines) disposed within, or entirely surrounded by, at least a portion of the proximal flexible tube section 20 and partial flared section 108. The proximal reinforcing region 200 comprises a distal section 202 that is skived or tapers downwardly in the proximal direction where it transitions into a middle non-tapered section 203 which, via a tapered section 206 that tapers proximally downwardly in the proximal direction to form a flat bottom connector section 204. The middle non-tapered section 203 forms a longitudinally semi-circular shape with an open top region as is best seen in the embodiments illustrated in FIGS. 9A-9D. The flat bottom connector section 204 comprises a notch or cutout 206 that is complementary in shape to the distal end of the flat ribbon section 112 discussed supra, such that the flat ribbon section 112 may be operationally interconnected with the cutout or notch 206 by a variety of means well known to the artisan including without limitation, welded, soldered, and snap-fit.

FIG. 8A illustrates one embodiment of the proximal reinforcing region 200 and showing the distal section 202 that is skived or tapers downwardly in the proximal direction to transition into the middle non-tapered section 203 and which comprises an open semicircular shape along its longitudinal length. At the proximal end, proximal reinforcing region 200 comprises a bottom connector section 204′ that may comprise a curvilinear shape to match the curvature of the surrounding polymer wall W. Bottom connector section 204′ may comprises, as shown, a cutout or notch 206.

In all embodiments comprising the proximal reinforcing region 200 the interconnection between flat ribbon section 112 and the cutout or notch 206 is shown as comprising two flat interconnecting structures, 112 and 204 such that flat ribbon section 112 fits within the cutout or notch 206 and secured by known means such as welding, soldering and/or snap-fit. However, as shown in FIG. 8A, the flat ribbon section 112 may in some embodiments be modified to conform to, or complement, the curvilinear shape of the wall of the elongated flexible tube and/or the middle non-tapered section 203. In this embodiment, the cutout or notch 206 and connector section may all comprise complementary curvatures to that of the curved ribbon section to facilitate a secured and operational engagement therebetween.

In some embodiments, the proximal reinforcing region 200 may be formed from the spiral laser-cut tube 118 and/or 118′ of FIGS. 4A and 4B. In other embodiments, the proximal reinforcing region 200 may be separately manufactured and then operationally connected with the flexible tube section 102 and flat ribbon section 112. The proximal reinforcing region 200 may, therefore, be used in connection with any of the embodiments of the flexible tube section 102 that are discussed supra.

The proximal reinforcing region 200 may be, as discussed, preferably surrounded by, or disposed within, a polymer coating or jacket, e.g., PTFE, or other material and, therefore, does not extend outwardly and proximally beyond the proximal-most end of the partial flared section 108. In other words, portions of the flexible tube 102, e.g., the proximal portion 120 of the flexible tube section 102 and the partial flared section 108 completely encompass and surround the proximal reinforcing region 200. In some embodiments, the proximal reinforcing region 200 may be embedded within the polymer material defining or surrounding and/or lining the proximal portion 120 of the flexible tube section 102 and partial flared section 108.

Alternatively, in some embodiments, the proximal reinforcing region 200 may be present as described above, but the resulting device comprises a partial cylinder 210, instead of a partial flared section 108, wherein the partial cylinder 200 does not taper outwardly at a proximal end, but does comprise a flattened lower surface 110′ that, similar to the flattened lower surface 110 of the partial flared section 108, is complementary in shape to the flat ribbon section 112 and configured for interconnecting flat ribbon section 112 and the notch or cutout 206.

FIGS. 9A-9D illustrate a proximal reinforcing region 200R for the proximal portion 120 of the flexible tube section 102, and one of the following semi-circular sections: a partial flared section 108, and a partial cylinder section 210; or one of the following complete semi-circular sections: a complete flared section 108′, and a complete cylinder section 210′, wherein in each embodiment, the proximal reinforcing region 200R does not extend proximally beyond the proximal-most end of the partial or complete semi-circular section, or the partial or complete cylinder section of the illustrated embodiments.

Thus, FIGS. 9A-9D illustrate an alternative embodiment of a proximal reinforcing region 200R, as described above, and further comprises a partial or semi-circular support ring 212 disposed at, or near, a distal end of the flat bottom connector section 204, or curved bottom connector 204′ (as shown), all of which remains completely surrounded by a portion of the flexible tube section 102 and/or partial flared section 108, complete flared section 108′, partial cylinder section 210 and/or complete cylinder section 210′. The partial or semi-circular support ring 212 may be embedded within the material of the proximal portion 120 of the flexible tube portion 102 and/or flare (partial and/or complete) and/or cylinder (partial and/or complete). The ring 212 extends circumferentially away from the flat bottom connector section 204 and/or the notch or cutout 206 on both sides of the notch of cutout 206 of proximal reinforcing section 200R to define an interrupted or partial or semi-circular ring 212 while retaining the connectivity between the notch and the flat ribbon. The partial ring 212 may be semi-circular in shape to match the radius or curvature of the polymer wall W and, in some embodiments, may comprise an upper surface that is parallel with central axis A, and in other embodiments may taper upward and outwardly in the distal direction to match the taper of the partial flared section 108, when present.

FIG. 9A shows the proximal reinforcing region 200R with partial or semi-circular support ring 212 attached thereto and a complete flared section 108′, i.e., without a flattened bottom portion 110. Alternatively, FIG. 9B provides the same proximal reinforcing region 200R with partial or semi-circular support ring 212 and a partial flared section 108 as discussed in connection with FIGS. 4A and 4B.

FIG. 9C illustrates the proximal reinforcing region 200R with partial or semi-circular support ring 212 attached thereto and, instead of a flared section (either complete or partial), a complete cylinder section 210′, i.e., without a flattened bottom portion 110, is provided.

FIG. 9D illustrates the proximal reinforcing region 200R with partial or semi-circular support ring 212 attached thereto and a partial cylinder 200R at the distal end of the flexible tube 200 with a flattened bottom section 110 to accommodate the flat ribbon 112 interconnection with the defined cutout or notch 206 of the proximal reinforcing region 200R.

In all cases, the support ring 212 does not extend distally beyond the proximal-most end of the semi-circular (partial or complete) section and, therefore is entirely surrounded by, or encased within, the polymer wall W of the proximal portion 120 of the flexible tube section 102 and/or the semi-circular section.

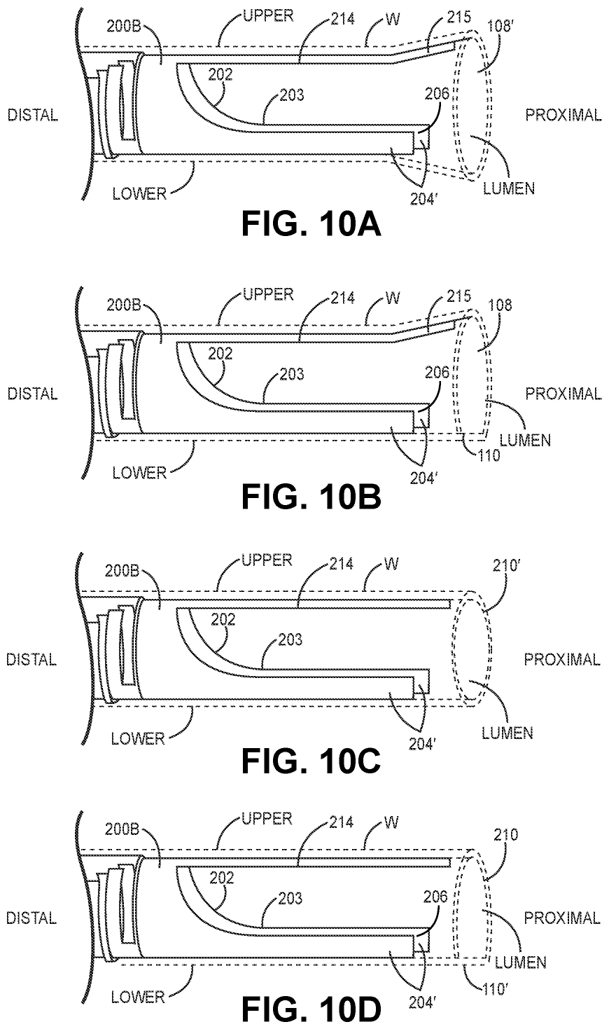

FIG. 10A illustrates an alternative proximal reinforcing section 200B, similar to that of FIGS. 8-8A, and further comprising an upper support beam 214 extending in the proximal direction away from the distal tapered section 202 along the flexible tube and the upper side of the flexible tube that is opposite that of the lower side comprising the cutout or notch 206 of the proximal reinforcing region 200B. A distal portion of the upper support beam 214 runs parallel with central axis A along the proximal portion 120 of the flexible tube section and, in some embodiments, may extend proximally along the semi-circular section, including in some flared (partial or complete) embodiments, an additional distal portion 215 that tapers outwardly in the distal direction to match the outward taper of the flare as shown. FIG. 10A illustrates a complete flared section 108′ at the proximal end, i.e., there is no flat bottom section. All elements of the proximal reinforcing region 200B, including the upper support beam, are surrounded by, or embedded within, the flexible tube and/or complete flare and do not extend proximally beyond the proximal-most end of the flared section 108′.

FIG. 10B illustrates an alternative proximal reinforcing section 200B to the embodiment of FIG. 10A, comprising the upper support beam 214 and a partial flared section 108 as in FIGS. 4A and 4B and therefore comprises the flattened bottom section 110 to accommodate the flat ribbon 112 and interconnection between flat ribbon 112 and the defined cutout 206 of proximal reinforcing region 200 as described supra. All elements of the proximal reinforcing region 200B, including the upper support beam, are surrounded by, or embedded within, the flexible tube and/or partial flared section 108 and do not extend proximally beyond the proximal-most end of the partial flared section 108.

FIG. 10C is further alternative proximal reinforcing section 200B comprising the upper support beam 214, wherein the complete flare 108′ of FIG. 10A is replaced by a complete cylinder 210′, wherein no portion of the proximal reinforcing section 200B, including the upper support beam 214 extends proximally beyond the proximal-most end of the complete cylinder 210′.

FIG. 10D illustrates an alternative proximal reinforcing region 200B comprising the upper support beam 214 and wherein the proximal reinforcing section 200 comprises a partial cylinder 210 with a flat bottom section 110 configured to accommodate the flat ribbon 112 and engagement thereof with the cutout 206 of the proximal reinforcing section 200B. As with the other embodiments, no portion of the proximal reinforcing section 200B, including the upper support beam 214 extends proximally beyond the proximal-most end of the partial complete cylinder 210.

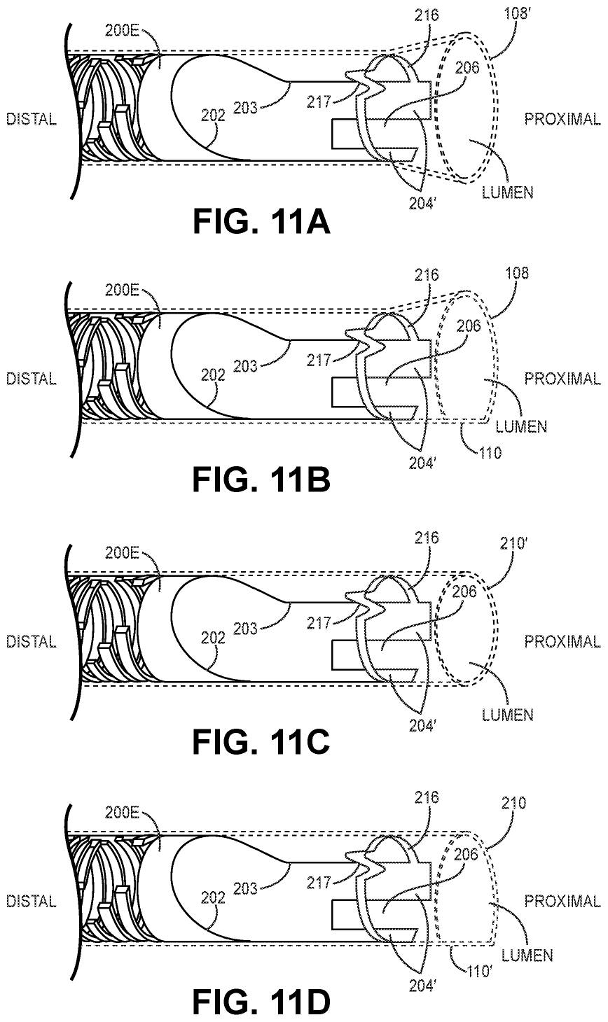

FIG. 11A is similar to the embodiment of FIG. 9A, wherein the proximal reinforcing section 200E comprising an expansion member 216 comprising an expansible portion 217 disposed along the circumference of the support ring 212 at or near the distal end of the proximal reinforcing region 200. Expansible portion 217 comprises a structure that elongates under sufficient force. For example, and without limitation, a spring, a shock absorber, a coil or coils may be implemented. FIGS. 11A-11D illustrate an exemplary z-shaped expansible portion 217. Thus, expansion member 216 is configured to change circumference as the exemplary z-shaped expansible portion 217 is forced to expand when a outwardly radially directed force is applied thereto, wherein the shape of the “z” becomes more linear and elongates, as in during translation of an interventional device. In some embodiments an inwardly directed radial force may be required to contract or collapse the expanded configuration back to an unexpanded configuration, or alternatively, the expansible portion 217 may be biased to return to the unexpanded configuration. Thus, the z-shaped expansible member embodiment 217 may move toward a more linear form as it expands or compresses, or collapses radially when subjected to an expansion or compression force that exceeds a base threshold force. In a preferred embodiment, the expansible portion 217 may be formed of a shape memory material such as Nitinol or the like with an undeformed resting configuration and a deformed, expanded configuration.

Alternatively, expansible portion 217 may be biased to an undeformed resting configuration, but may be expanded if an at least partially outwardly radially directed force is applied that overcomes the biasing force. Expansion member 216 with expansible portion 217 thus provides a way to more easily translate an interventional device into and through the lumen of the flexible tube 102. The expansion member 216, and expansible member 217 may be surrounded by the flexible tube and/or embedded therein and does not extend proximally beyond the proximal-most end of the complete flare section 108′ of FIG. 11A.

FIG. 11B comprises an alternative embodiment of the proximal reinforcing section 200E of FIG. 11A, wherein the complete flare section 108′ of FIG. 11A is replaced by a partial flare section 108 and wherein the flattened bottom section 110 is provided.

FIG. 11C provides another embodiment of the proximal reinforcing section 200E, similar to that of FIG. 11A, but wherein the complete flare section 108′ of FIG. 11A is replaced with a complete cylinder 210′.

FIG. 11D provides another embodiment of the proximal reinforcing section 200E, similar to that of FIG. 11C, but, now comprising a partial cylinder 210 with flattened lower surface 110′.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A guide extension catheter comprising:
a flexible tube section having
a flexible, reinforced portion comprising one or more of the group consisting of: a laser-cut tube, a braid, and at least two oppositely wound coils, wherein the flexible tube section is coated with a polymer that defines a wall,
a proximal reinforcing section disposed proximal to the flexible, reinforced portion and disposed within the wall of the flexible tube section, the proximal reinforcing section comprising:
a skived distal section that tapers downwardly in the proximal direction,
a middle non-tapered section with a longitudinally open semi-circular shape and a proximal end that tapers downwardly in the proximal direction;
a semi-circular section formed by the polymer defining the wall of the flexible
tube section and disposed proximal to the proximal reinforcing section and comprising a flattened bottom section;
a flat ribbon section connected with a proximal portion of the proximal reinforcing section portion and extending proximally along the flattened bottom section of the semi-circular section; and
a push rod operatively connected with a proximal portion of the flat ribbon section, the push rod comprising a proximal handle,
wherein the proximal reinforcing section does not extend proximally beyond a proximal end of the semi-circular section.

2. (canceled)

3. The guide extension catheter of claim 1, wherein the proximal reinforcing section comprises a flat or curvilinear bottom connector section and wherein the flat ribbon section is connected to the flat or curvilinear bottom connector section.

4. The guide extension catheter of claim 3, wherein
a proximal end of flat or curvilinear bottom connector section defines a cutout that is complementary in shape with a shape of a distal end of the flat ribbon section, whereby the flat ribbon section is operatively connected within the cutout and thereby connected with the proximal reinforcing section.

5. The guide extension catheter of claim 1, wherein the connected the flat ribbon section does not reduce an inner diameter of the flexible tube section.

6. The guide extension catheter of claim 4, wherein the connected flat ribbon section does reduce an inner diameter of the flexible tube section.

7. The guide extension catheter of claim 1, wherein the semi-circular section comprises a partial cone-shaped flared shape.

8. The guide extension catheter of claim 1, wherein the semi-circular section comprises a partial cylinder shape.

9. The guide extension catheter of claim 1, wherein the laser-cut tube comprises spiral cuts, wherein the flexible tube section comprises a central longitudinal axis,
wherein the spiral cuts comprise a pitch and an angle relative to the central axis, and
wherein the pitch and angle are constant moving from a distal to a proximal direction along the laser-cut tube.

10. The guide extension catheter of claim 1, wherein the laser-cut tube comprises spiral cuts, wherein the flexible tube section comprises a central longitudinal axis,
wherein the spiral cuts comprise a pitch and an angle relative to the central axis, and
wherein the pitch and angle are not constant moving from a distal to a proximal direction along the laser-cut tube.

11. The guide extension catheter of claim 10, wherein the pitch of the spiral cuts becomes longer moving from a distal to a proximal direction along the laser-cut tube.

12. A guide extension catheter comprising:
a flexible tube section having
a flexible, reinforced portion comprising one or more of the group consisting of: a laser-cut tube, a braid, and at least two oppositely wound coils, wherein the flexible tube section is coated with a polymer that defines a wall;
a proximal reinforcing section disposed proximal to the flexible, reinforced portion and comprising:
a skived distal section that tapers downwardly in the proximal direction,
a middle non-tapered section with a longitudinally open semi-circular shape,
a flat or curvilinear bottom connector section with a proximal end that defines a cutout, and
a semi-circular support ring connected with the flat or curvilinear bottom connector section at a proximal end of the proximal reinforcing section,
wherein the proximal reinforcing section is disposed within the wall of the flexible tube section;
a semi-circular section formed by the polymer defining the wall of the flexible tube section and disposed proximal to the proximal reinforcing section and comprising a flattened bottom section, and
a flat ribbon section extending along the flattened bottom section of the semi-circular section, wherein the flat ribbon section is operatively connected within the cutout defined by the flat or curvilinear bottom section; and
a push rod operatively connected with a proximal portion of the flat ribbon section, the push rod comprising a proximal handle,
wherein the proximal reinforcing section does not extend proximally beyond a proximal end of the semi-circular section.

13. The guide extension catheter of claim 12, wherein the proximal semi-circular section comprises a partial cone-shaped flared shape.

14. The guide extension catheter of claim 12, wherein the proximal semi-circular section comprises a partial cylinder shape.

15. The guide extension catheter of claim 12, wherein the semi-circular support ring comprises an expansion member and further comprising an expansible portion, wherein the expansible portion is configured to achieve an undeformed unexpanded configuration and configured to expand to a larger deformed configuration when subjected to a radially outward force.

16. The guide extension catheter of claim 15, wherein the expansible portion is biased to retain the undeformed configuration.

17. The guide extension catheter of claim 15, wherein the expansible portion comprises a shape memory material.

18. The guide extension catheter of claim 15, wherein the expansion member comprises a z-shape in the undeformed configuration.

19. A guide extension catheter comprising:
a flexible tube section having
a flexible, reinforced portion comprising one or more of the group consisting of: a laser-cut tube; a braid; and at least two oppositely wound coils, wherein the flexible tube section is coated with a polymer that defines a wall;
a proximal reinforcing section disposed proximal to the proximal flexible tube portion and comprising:
a skived distal section that tapers downwardly in the proximal direction,
a middle non-tapered section with a longitudinally open semi-circular shape,
a flat or curvilinear bottom connector section with a proximal end that defines a cutout,
wherein the proximal reinforcing section is disposed within the wall of the flexible tube section; and
a complete cone-shaped flared section formed by the polymer that defines the wall of the flexible tube section and disposed proximal to the proximal reinforcing section, the complete cone-shaped flared section comprising longitudinal cross sections that are completely circular;
a flat ribbon section connected with a proximal portion of the flexible, reinforced portion of the flexible tube section; and
a push rod operatively connected with a proximal portion of the flat ribbon section, the push rod comprising a proximal handle,
wherein the proximal reinforcing section does not extend proximally beyond a proximal end of the complete cone-shaped flared section.

20. (canceled)
